(19) United States
(12) Patent Application Publication
(10) Pub. No.: US 2014/0248230 A1
(43) Pub. Date: Sep. 4, 2014

(54) HAIR CARE TREATMENT AGENT

(71) Applicant: Henkel AG & Co. KGaA, Duesseldorf (DE)

(72) Inventors: Edith von Aspern, Hanstedt (DE); Jens Delowsky, Norderstedt (DE); Joerg Kahre, Leichlingen (DE)

(73) Assignee: Henkel AG & Co. KGaA, Duesseldorf (DE)

(21) Appl. No.: 14/278,258

(22) Filed: May 15, 2014

Related U.S. Application Data
(63) Continuation of application No. PCT/EP2012/071122, filed on Oct. 25, 2012.

(30) Foreign Application Priority Data
Nov. 17, 2011 (DE) 10 2011 086 537.3

(51) Int. Cl. A61K 8/49; A61Q 5/00; A61K 8/44; A61K 8/41; A61K 8/81; A61K 8/25

(52) U.S. Cl. CPC A61K 8/494; A61K 8/8158; A61K 8/25; A61K 8/44; A61K 8/416; A61Q 5/00
USPC 424/70.122; 424/70.16

(57) ABSTRACT

Hair treatment agents include at least one quaternary ammonium compound according to formula (I), in which n denotes an integer from 10 to 24 and X⁻ denotes an anion, at least one quaternary ammonium compound according to formula (II) in which m denotes an integer from 10 to 24 and X⁻ denotes an anion, at least one quaternary ammonium compound of formula (III) in which R and R′ independently of one another each denote a saturated or unsaturated, linear or branched hydrocarbon residue having a chain length of 18 to 30 carbon atoms and X″ denotes an anion, and at least one cationic copolymer, including monomers of formula (IV) and monomers of formula (V) in which R¹ to R⁹ independently of one another denote hydrogen or C₁₋₄ alkyl (R⁶, R⁷, R⁸ or R⁹ denotes C₁₋₄ alkyl), n denotes integers from 1 to 8 and A⁻ denotes a physiologically acceptable anion

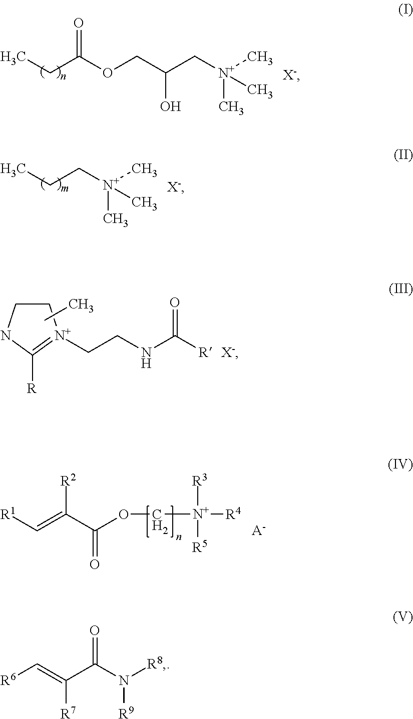

### FIELD OF THE INVENTION

The present invention generally relates to cosmetic hair treatment agents based on a special combination of active ingredients, to a method using the agents, and to the use of the agents to care for and improve the combability of keratinic fibers.

### BACKGROUND OF THE INVENTION

Not least because of the heavy loading on the hair, resulting for example from coloring or permanent waving and from washing the hair with shampoos as well as from environmental pollution, the importance of care products having as long-lasting an effect as possible is growing.

However, the known active ingredients cannot cover all requirements to an adequate extent. There is therefore still a demand for active ingredients or for combinations of active ingredients for cosmetic agents having good care properties and good biodegradability. In formulations including dyes and electrolytes in particular there is a need for additional caring active ingredients that are able to be incorporated without difficulty into known formulations.

Hair treatment agents including one or more quaternary ammonium compounds (QACs) as care substances are widely known in the prior art.

It is therefore desirable to further develop these combinations of active ingredients and to improve them, in particular with regard to the combability and softness (feel) of keratinic fibers. It is further desirable to provide a combination of active ingredients that delivers outstanding care performance while reducing the amounts used overall.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It has now been found that the addition of certain polymers to a certain combination of three different QACs still further improves the combability, shine and elasticity. Furthermore, the washing resistance of colored hair was also able to be improved. Surprisingly, the feel of the keratinic fibers in particular was exceptionally positively influenced. The stated effects surprisingly were clearly apparent even with a reduced total amount overall of cationic active substances.

In accordance with this finding, a hair treatment agent includes at least one quaternary ammonium compound according to formula I

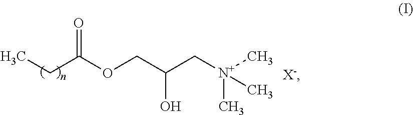

in which n denotes an integer from 10 to 24 and X⁻ denotes an anion;
at least one quaternary ammonium compound according to formula II

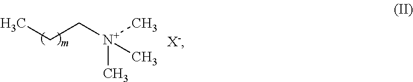

in which m denotes an integer from 10 to 24 and X⁻ denotes an anion;
at least one quaternary ammonium compound of formula III

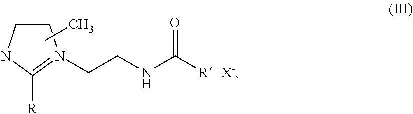

in which R and R′ independently of one another each denote a saturated or unsaturated, linear or branched hydrocarbon residue having a chain length of 16 to 30 carbon atoms and X⁻ denotes an anion; and
at least one cationic copolymer, including monomers of formula (IV) and monomers of formula (V)

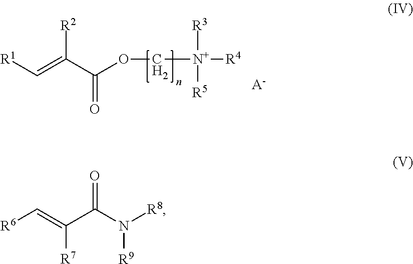

in which R¹ to R⁹ independently of one another denote hydrogen, C₁₋₄ alkyl such as methyl, ethyl, propyl, isopropyl, butyl, isobutyl or tert-butyl, with the proviso that at least one of the residues R⁶, R⁷, R⁸ or R⁹ denotes C₁₋₄ alkyl, n denotes integers from 1 to 8 and A⁻ denotes a physiologically acceptable anion.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention provides in a first embodiment hair treatment agents including
a) at least one quaternary ammonium compound according to formula I

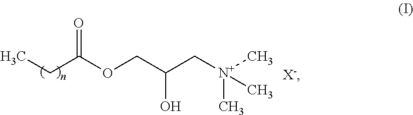

in which n denotes an integer from 10 to 24 and X⁻ denotes an anion,
b) at least one quaternary ammonium compound according to formula II

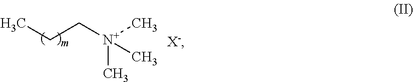

in which m denotes an integer from 10 to 24 and X⁻ denotes an anion,
c) at least one quaternary ammonium compound of formula III

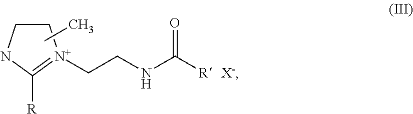

in which R and R′ independently of one another each denote a saturated or unsaturated, linear or branched hydrocarbon residue having a chain length of 18 to 30 carbon atoms and X⁻ denotes an anion,
d) at least one cationic copolymer, including monomers of formula (IV) and monomers of formula (V)

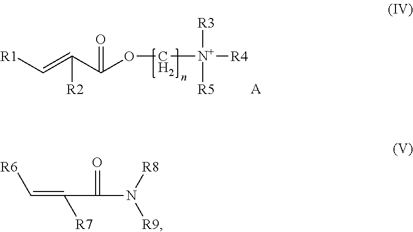

in which R¹ to R⁹ independently of one another denote hydrogen, C₁₋₄ alkyl such as methyl, ethyl, propyl, isopropyl, butyl, isobutyl or tert-butyl, with the proviso that at least one of the residues R⁶, R⁷, R⁸ or R⁹ denotes C₁₋₄ alkyl, n denotes integers from 1 to 8 and A⁻ denotes a physiologically acceptable anion.

Hair treatment agents within the meaning of the present invention are for example hair coloring agents, bleaching agents, hair shampoos, hair conditioners, conditioning shampoos, hair sprays, hair rinses, hair masks, hair packs, hair tonics, permanent wave fixing solutions, hair coloring shampoos, hair coloring agents, hair fixing agents, hair setting agents, hair styling preparations, blow-drying lotions, styling mousses, hair gels, hair waxes or combinations thereof Preferred agents according to the invention are shampoos, conditioning agents or hair tonics.

As ingredient a) the agents according to the invention include at least one quaternary ammonium compound according to formula I

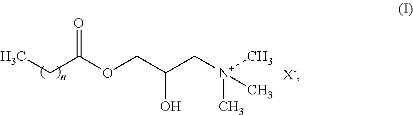

in which n denotes an integer from 10 to 24 and X⁻ denotes an anion. Under INCI nomenclature compounds of formula (I) are known as Alkanoyl PG Trimonium salts. Preferred values for n are the numbers 16, 18 and 20, preferred anion ions are chloride and methosulfate.

The QACs of formula (I) are included in the compositions according to the invention by preference in amounts from 0.01 to 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the total composition of the individual agent.

Regarding the storage stability of the compositions according to the invention, hair treatment agents according to the invention are particularly preferred which include—relative to their weight—0.1 to 20 wt. %, preferably 0.2 to 10 wt. %, more preferably 0.3 to 7.5 wt. %, still more preferably 0.4 to 5 wt. % and in particular 0.5 to 2.5 wt. % of at least one quaternary ammonium compound of formula I in which n denotes the number 20 and A⁻ denotes chloride (INCI name: Behenoyl PG Trimonium Chloride).

As ingredient b) the agents according to the invention include at least one quaternary ammonium compound according to formula II

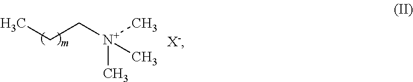

in which m denotes an integer from 10 to 24 and X⁻ denotes an anion. Under INCI nomenclature compounds of formula (II) are known as Alkane Trimonium salts. Preferred values for n are the numbers 16, 18 and 20, preferred anion ions are chloride and methosulfate.

The QACs of formula (II) are included in the compositions according to the invention by preference in amounts from 0.01 to 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the total composition of the individual agent.

Regarding the care performance, in particular the detangling ability, combability and feel of hair treated with compositions according to the invention, hair treatment agents according to the invention are preferred which include—relative to their weight—0.1 to 15 wt. %, preferably 0.2 to 10 wt. %, more preferably 0.3 to 7.5 wt. %, still more preferably 0.4 to 5 wt. % and in particular 0.5 to 2.5 wt. % of at least one quaternary ammonium compound of formula II in which n denotes the number 20 and A⁻ denotes chloride (INCI name: Behentrimonium Chloride).

As ingredient c) the agents according to the invention include at least one quaternary ammonium compound of formula III

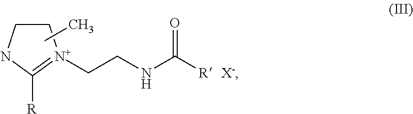

in which R and R′ independently of one another each denote a saturated or unsaturated, linear or branched hydrocarbon residue having a chain length of 16 to 30 carbon atoms and X⁻ denotes an anion.

Particularly preferred agents according to the invention include as ingredient c) at least one quaternary imidazoline derivative having at least two long fatty residues according to formula IIIa

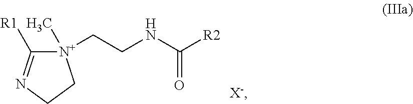

in which the residues R¹ and R² independently of one another each denote a saturated or unsaturated, linear or branched hydrocarbon residue having a chain length of 16 to 30 carbon atoms and X⁻ denotes an anion.

The agents according to the invention preferably include compounds of formula (IIIa) in which R¹═R². The chain length of the residues R is at least 16 carbon atoms. Compounds having a chain length of 16, 18 or 20 carbon atoms and particularly preferably having 16 or 18 carbon atoms are preferred. A most particularly preferred compound of formula Ma has alkyl residues derived from tallow alcohol as the residues R¹ or R². A commercial product of this chain length is known for example under the name Quaternium-87.

The counterion X⁻ in formula (I) is preferably methosulfate, but the halides such as chloride, fluoride, bromide and also phosphates are also suitable as counterions.

The imidazolines of formulae (III) or (IIIa) are included in the compositions according to the invention by preference in amounts from 0.01 to 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the total composition of the individual agent.

Most particularly preferred hair treatment agents according to the invention are characterized in that they include—relative to their weight—0.1 to 15 wt. %, preferably 0.2 to 10 wt. %, more preferably 0.3 to 7.5 wt. %, still more preferably 0.4 to 5 wt. % and in particular 0.5 to 2.5 wt. % of at least one quaternary ammonium compound of formula III in which R and R′ independently of one another are selected from stearyl and oleoyl residues and A⁻ denotes methosulfate (INCI name: Quatemium-87).

As ingredient d) the agents according to the invention include at least one cationic copolymer. The cationic copolymer is synthesized from monomers of formulae (IV) and (V),

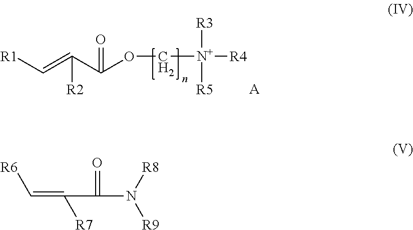

in which R¹ to R⁹ independently of one another denote hydrogen, C₁-C₄ alkyl such as methyl, ethyl, propyl, isopropyl, butyl, isobutyl or tert-butyl, with the proviso that at least one of the residues R⁶, R⁷, R⁸ or R⁹ denotes C₁-C₄ alkyl, n is an integer and denotes numbers from 1 to 8, and A⁻ denotes a physiologically acceptable anion such as fluoride, chloride, bromide, iodide, hydrogen sulfate or methosulfate.

R¹ is preferably hydrogen or a methyl group, particularly preferably a methyl group. R² is preferably hydrogen or a C₁-C₄ alkyl group, particularly preferably hydrogen. R³, R⁴ and R⁵ are preferably independently of one another hydrogen or a C₁-C₄ alkyl group, in particular a methyl group. R³, R⁴ and R⁵ are particularly preferably the same and highly preferably methyl. The number n is preferably an integer and denotes numbers from 1 to 7, more preferably from 1 to 5, highly preferably 1, 2, 3 or 4 and in particular 2. R⁶ is preferably hydrogen or methyl. R⁷ is preferably hydrogen, methyl or ethyl, particularly preferably methyl. R⁸ and R⁹ are preferably the same and denote hydrogen, methyl or C₁-C₄ alkyl, particularly preferably hydrogen or methyl. Most preferably the monomer of formula (V) denotes dimethylacrylamide.

The cationic copolymer d) preferably includes 20 to 95 wt. % of monomers of formula (IV) and 5 to 50 wt. % of monomers of formula (V).

Particularly preferred cationic copolymers d) include 40 to 90 wt. % of monomer (IV) and 10 to 40 wt. % of monomer (V).

The cationic copolymer d) can moreover be crosslinked. In this case crosslinking is brought about by means of the conventional crosslinking agents such as for example allylacrylamide, allylmethacrylamide, tetraallylammonium chloride or N,N′-methylenebisacrylamide, in amounts up to 500 ppm.

The cationic copolymer d) can moreover be present as a solution, suspension or dispersion in suitable cosmetic media, such as for example propylene glycol, glycerol, paraffin, isoparaffin, propylene glycols and esters of propylene glycol such as for example propylene glycol dicaprylate/dicaprate, or PPG-1 Trideceth-6 and others.

One example of a particularly preferred copolymer d) is the commercial product Tinovis® CD from Ciba.

The cationic copolymer d) is preferably used in the agents according to the invention in amounts from 0.1 to 15 wt. % and particularly preferably in amounts from 0.1 to 5.0 wt. %, relative to the weight of the agent.

Most particularly preferred hair treatment agent according to the invention are characterized in that they include—relative to their weight—0.1 to 15 wt. %, preferably 0.2 to 10 wt. %, more preferably 0.3 to 7.5 wt. %, still more preferably 0.4 to 5 wt. % and in particular 0.5 to 2.5 wt. % of at least one cationic copolymer including monomers of formula (IV) and monomers of formula (V), in which R¹═R⁶═R⁷═—H and R²═R³═R⁴═R⁵═R⁸═R⁹═—CH₃, n denotes the number 2 and A′ denotes chloride (INCI name: Dimethylacrylamide/Ethyltrimonium Chloride Methacrylate Copolymer).

The combination of four active ingredients in the agents according to the invention can be supplemented with further auxiliary substances. Thus it has been found for example that esters of long-chain alcohols (fatty alcohols) with longer-chain monocarboxylic acids still further increase the fullness and softness of the hair when they are incorporated into the agents according to the invention. Particularly preferred hair treatment agents according to the invention are characterized in that they additionally include esters of coconut fatty alcohols with octanoic acid.

Other emollients such as dicaprylyl carbonate, dicaprylyl ether, propylheptyl caprylate, etc., also show these effects, but they are particularly pronounced in the cocoalkyl esters of octanoic acid.

Most particularly preferred agents according to the invention include
0.1 to 15 wt. % of Behenoyl PG Trimonium Chloride and
0.1 to 15 wt. % of Behentrimonium Chloride and
0.1 to 15 wt. % of Quaternium-87 and
0.1 to 15 wt. % of Dimethylacrylamide/Ethyltrimonium Chloride Methacrylate Copolymer and
0.1 to 10 wt. % of esters of coconut fatty alcohols with octanoic acid.

The use of spreading agents has likewise proved particularly advantageous, since the care effects of the combination according to the invention are brought to bear even more uniformly as a result. Particularly preferred hair treatment agents according to the invention are characterized in that they additionally include—relative to their weight—0.1 to 20 wt. %, preferably 0.2 to 10 wt. %, more preferably 0.3 to 7.5 wt. %, still more preferably 0.4 to 5 wt. % and in particular 0.5 to 2.5 wt. % of isopropyl myristate.

Most particularly preferred agents according to the invention include
0.1 to 15 wt. % of Behenoyl PG Trimonium Chloride and
0.1 to 15 wt. % of Behentrimonium Chloride and
0.1 to 15 wt. % of Quaternium-87 and
0.1 to 15 wt. % of Dimethylacrylamide/Ethyltrimonium Chloride Methacrylate Copolymer and
0.5 to 2.5 wt. % of isopropyl myristate.

Most particularly preferred agents include both spreading agents and fatty substance(s), preferably esters of octanoic acid with long-chain fatty alcohols.

Most particularly preferred agents according to the invention include
0.1 to 15 wt. % of Behenoyl PG Trimonium Chloride and
0.1 to 15 wt. % of Behentrimonium Chloride and
0.1 to 15 wt. % of Quaternium-87 and
0.1 to 15 wt. % of Dimethylacrylamide/Ethyltrimonium Chloride Methacrylate Copolymer and
0.1 to 10 wt. % of esters of coconut fatty alcohols with octanoic acid and
0.5 to 2.5 wt. % of isopropyl myristate.

The agents according to the invention can include further ingredients.

The agents according to the invention include amphoteric surfactant(s) to particular advantage. Ampholytic surfactants and emulsifiers are understood to be surface-active compounds which in addition to a C₈-C₂₄ alkyl or acyl group include at least one free amino group and at least one —COOH or —SO₃H group and are capable of forming internal salts. Examples of suitable ampholytic surfactants are N-alkyl glycines, N-alkyl aminopropionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytic surfactants are N-cocoalkyl aminopropionate, cocoacyl aminoethyl aminopropionate and C₁₂-C₁₈ acyl sarcosine.

Preferred hair treatment agents according to the invention are characterized in that they include 1 to 30 wt. %, preferably 6 to 25 wt. %, more preferably 7 to 20 wt. %, still more preferably 8 to 15 wt. % and in particular 10 to 12.5 wt. % of amphoteric surfactant(s).

Particularly preferred hair treatment agents according to the invention are characterized in that they include amphoteric surfactant(s) from the groups of
N-alkyl glycines,
N-alkyl propionic acids,
N-alkyl aminobutyric acids,
N-alkyl iminodipropionic acids,
N-hydroxyethyl-N-alkylamidopropyl glycines,
N-alkyl taurines,
N-alkyl sarcosines,
2-alkyl aminopropionic acids each having approximately 8 to 24 C atoms in the alkyl group,
alkyl aminoacetic acids each having approximately 8 to 24 C atoms in the alkyl group,
N-cocoalkyl aminopropionate,
cocoacyl aminoethyl aminopropionate,
C₁₂-C₁₈ acyl sarcosine,
N-alkyl-N,N-dimethylammonium glycinates, for example cocoalkyl dimethylammonium glycinate,
N-acyl aminopropyl-N,N-dimethylammonium glycinates, for example cocoacyl aminopropyl dimethylammonium glycinate,
2-alkyl-3-carboxymethyl-3-hydroxyethyl imidazolines, each having 8 to 18 C atoms in the alkyl or acyl group,
cocoacyl aminoethyl hydroxyethyl carboxymethyl glycinate,
the compounds known under the INCI name Cocamidopropyl Betaine,
the compounds known under the INCI name Disodium Cocoamphodiacetate, preferred agents including the amphoteric surfactant(s) in amounts from 0.5 to 9 wt. %, preferably from 0.75 to 8 wt. % and in particular from 1 to 7.5 wt. %, relative in each case to the total agent.

Particularly preferred hair treatment agents include as amphoteric surfactants betaines of the formula (Bet-I)

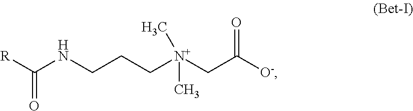

in which R denotes a straight-chain or branched, saturated or mono- or polyunsaturated alkyl or alkenyl residue having 8 to 24 carbon atoms.

Under INCI nomenclature these surfactants are known as Amidopropylbetaines, wherein the representatives derived from coconut fatty acids are preferred and are known as Cocoamidopropylbetaines. Surfactants of the formula (Bet-I) which are a mixture of the following representatives are particularly preferably used according to the invention:
H₃C-(CH₂)₇—C(O)—NH—(CH₂)₃N⁺(CH₃)₂CH₂COO⁻
H₃C-(CH₂)₉—C(O)—NH—(CH₂)₃N⁺(CH₃)₂CH₂COO⁻
H₃C-(CH₂)₁₁—C(O)—NH—(CH₂)₃N⁺(CH₃)₂CH₂COO⁻
H₃C-(CH₂)₁₃—C(O)—NH—(CH₂)₃N⁺(CH₃)₂CH₂COO⁻
H₃C-(CH₂)₁₅—C(O)—NH—(CH₂)₃N⁺(CH₃)₂CH₂COO⁻
H₃C-(CH₂)₇—CH═CH—(CH₂)₇-C(O)—NH—(CH₂)₃N⁺(CH₃)₂CH₂COO⁻

Surfactants of the formula (Bet-I) are particularly preferably used within relatively narrow quantity ranges. Agents according to the invention are preferred here which—relative to their weight—include 0.25 to 8 wt. %, more preferably 0.5 to 7 wt. %, more preferably 0.75 to 6.5 wt. % and in particular 1 to 5.5 wt. % of surfactant(s) of the formula (Bet-I).

In addition to the amphoteric surfactant(s) of the formula (Bet-I) or in their place, the hair treatment agents according to the invention can include to particular advantage as amphoteric surfactants betaines of the formula (Bet-II)

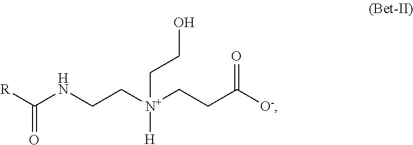

in which R denotes a straight-chain or branched, saturated or mono- or polyunsaturated alkyl or alkenyl residue having 8 to 24 carbon atoms.

Under INCI nomenclature these surfactants are known as Amphoacetates, wherein the representatives derived from coconut fatty acids are preferred and are known as Cocoamphoacetates.

For production reasons surfactants of this type always also include betaines of the formula (Bet-IIa)

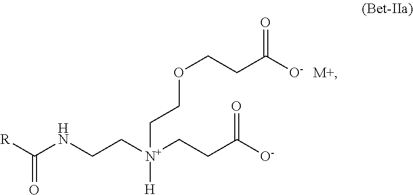

in which R denotes a straight-chain or branched, saturated or mono- or polyunsaturated alkyl or alkenyl residue having 8 to 24 carbon atoms and M denotes a cation.

Under INCI nomenclature these surfactants are known as Amphodiacetates, wherein the representatives derived from coconut fatty acids are preferred and are known as Cocoamphodiacetates.

Surfactants of the formula (Bet-II) which are a mixture of the following representatives are particularly preferably used according to the invention:
H₃C-(CH₂)₇—C(O)—NH—(CH₂)₂NH⁺(CH₂CH₂OH)CH₂CH₂COO⁻
H₃C-(CH₂)₉—C(O)—NH—(CH₂)₂NH⁺(CH₂CH₂OH)CH₂CH₂COO⁻
H₃C-(CH₂)₁₁—C(O)—NH—(CH₂)₂NH⁺(CH₂CH₂OH)CH₂CH₂COO⁻
H₃C-(CH₂)₁₃—C(O)—NH—(CH₂)₂NH⁺(CH₂CH₂OH)CH₂CH₂COO⁻
H₃C-(CH₂)₁₅—C(O)—NH—(CH₂)₂NH⁺(CH₂CH₂OH)CH₂Ch₂COO⁻
H₃C-(CH₂)₇—CH═CH—(CH₂)₇—C(O)—NH—(CH₂)₂NH⁺(CH₂CH₂OH)CH₂CH₂COO⁻

Surfactants of the formula (Bet-II) are particularly preferably used within relatively narrow quantity ranges. Agents according to the invention are preferred here which—relative to their weight—include 0.25 to 8 wt. %, more preferably 0.5 to 7 wt. %, more preferably 0.75 to 6.5 wt. % and in particular 1 to 5.5 wt. % of surfactant(s) of the formula (Bet-II).

In addition to the amphoteric surfactants or in their place the agents according to the invention can also include non-ionic surfactants and/or cationic surfactants (see above).

Particularly preferred non-ionic surfactants are alkyl polyglycosides. Alkyl polyglycosides (APGs) are non-ionic surfactants produced entirely from sustainable raw materials (sugar structural units, predominantly glucose, e.g. from corn starch, and fatty alcohol, e.g. from coconut oil). Alkyl polyglycosides are obtainable by acid-catalyzed reaction (Fischer reaction) of sugars, in particular glucose (or starch), or of butyl glycosides with fatty alcohols.

This gives rise to complex mixtures of alkyl monoglucoside (alkyl α-D and β-D glucopyranoside and small proportions of alkyl glucofuranoside), alkyl diglucosides (isomaltosides, maltosides, etc.) and alkyl oligoglucosides (maltotriosides, maltotetraosides, etc.). The average degree of polymerization of commercial products whose alkyl residues are in the range from C8 to C16, is 1.2 to 1.5.

Alkyl polyglycosides corresponding to the general formula RO—(Z)ₓ in which R denotes alkyl, Z denotes sugar and x denotes the number of sugar units, are preferably used according to the invention.

Alkyl polyglycosides in which R consists
substantially of C₈ and C₁₀ alkyl groups,
substantially of C₁₂ and C₁₄ alkyl groups,
substantially of C₈ to C₁₆ alkyl groups or
substantially of C₁₂ to C₁₆ alkyl groups or
substantially of C₁₆ to C₁₈ alkyl groups
   are particularly preferred.

Any mono- or oligosaccharides can be used as the sugar structural unit Z. Sugars having 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar structural units are glucose, fructose, galactose, arabinose and sucrose. Preferred hair treatment agents according to the invention are thus characterized in that they include—relative to their weight—0.1 to 20 wt. %, preferably 1 to 10 wt. % and in particular 2 to 8 wt. % of alkyl polyglycoside(s) of the formula

H₃C—(CH₂)ₙ—O—(Z)ₓ

in which n denotes values from 5 to 21, preferably from 7 to 19, particularly preferably from 9 to 17 and in particular from 11 to 13, and k denotes values from 1.1 to 1.8, preferably from 1.2 to 1.5, and Z denotes a sugar structural unit from the group comprising glucose, fructose, galactose, arabinose, ribose, xylose, lyxose, allose, altrose, mannose, glucose, idose, talose and sucrose.

Glucose is a particularly preferred sugar structural unit (Z), such that preferred hair treatment agents according to the invention are characterized in that they include—relative to their weight—0.1 to 15 wt. %, preferably 1 to 10 wt. % and in particular 2 to 8 wt. % of alkyl polyglucoside(s) of the formula

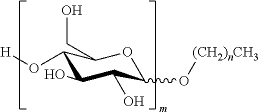

in which n denotes values from 5 to 21, preferably from 7 to 19, particularly preferably from 9 to 17 and in particular from 11 to 13, and m denotes numbers from 1.1 to 1.8, preferably from 1.2 to 1.5.

The alkyl polyglycosides for use according to the invention include on average 1.1 to 5 sugar units. Alkyl polyglycosides having x values from 1.1 to 2.0 are preferred. Alkyl glycosides in which x is 1.1 to 1.8 are most particularly preferred.

Most particularly preferred alkyl polyglucosides are those in which the alkyl residue is a lauryl residue. In the case of mixtures of substances from native sources, those sources having a high proportion of C12 fatty acids, in particular coconut fatty acids, are preferred. Particularly preferred hair treatment agents according to the invention are thus characterized in that they include—relative to their weight—0.1 to 15 wt. %, preferably 1 to 10 wt. % and in particular 2 to 8 wt. % of alkyl polyglucoside(s), in which n denotes the value 11, m denotes values from 1.1 to 1.8, preferably from 1.2 to 1.5.

The care effects of the agents according to the invention can be further strengthened by the use of certain care substances. These are preferably selected from certain groups of care substances known per se, since in terms of their formulation and their care effect these care substances harmonize superbly with the combination according to the invention.

Hair treatment agents that are preferred according to the invention are characterized in that they additionally include care substance(s)—relative to their weight—in amounts from 0.001 to 10 wt. %, preferably 0.005 to 7.5 wt. %, particularly preferably 0.01 to 5 wt. % and in particular 0.05 to 2.5 wt. %, preferred care substance(s) being selected from the group comprising
i. L-carnitine and/or salts thereof;
ii. taurine and/or salts thereof;
iii. niacinamide;
iv. ubiquinone;
v. ectoine.

L-Carnitine (IUPAC name (R)-(3-Carboxy-2-hydroxypropyl)-N,N,N-trimethylammonium hydroxide), is a naturally occurring, vitamin-like substance.

As a betaine, L-carnitine can form addition compounds and double salts. L-Carnitine derivatives that are preferred according to the invention are selected in particular from acetyl L-carnitine, L-carnitine fumarate, L-carnitine citrate, lauroyl L-carnitine and particularly preferably L-carnitine tartrate. The specified L-carnitine compounds are available for example from Lonza GmbH (Wuppertal, Germany).

Preferred hair treatment agents according to the invention are characterized in that they include—relative to their weight—0.001 to 10 wt. %, preferably 0.005 to 7.5 wt. %, particularly preferably 0.01 to 5 wt. % and in particular 0.05 to 2.5 wt. % of L-carnitine or L-carnitine derivatives, preferred L-carnitine derivatives being selected from acetyl L-carnitine, L-carnitine fumarate, L-carnitine citrate, lauroyl L-carnitine and in particular L-carnitine tartrate.

A further preferred care substance which can be used, and which has activating properties, is taurine. Hair treatment agents that are preferred according to the invention include—relative to their weight—0.01 to 15 wt. %, preferably 0.025 to 12.5 wt. %, particularly preferably 0.05 to 10 wt. %, more preferably 0.1 to 7.5 wt. % and in particular 0.5 to 5 wt. % of taurine (2-aminoethanesulfonic acid).

A further preferred group of care substances in the agents according to the invention are vitamins, provitamins or vitamin precursors. These are described below:

The group of substances classed as vitamin A includes retinol (vitamin Ai) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention include the vitamin A component preferably in amounts from 0.05 to 1 wt. %, relative to the total preparation.

The vitamin B group or vitamin B complex includes inter alia
Vitamin Bi (thiamine)
Vitamin B2 (riboflavin)
Vitamin B3. The compounds nicotinic acid and nicotinic acid amide (niacinamide) are often included under this term. Preferred according to the invention is nicotinic acid amide, which is preferably included in the agents used according to the invention in amounts from 0.05 to 1 wt. %, relative to the total agent.
Vitamin B₅ (pantothenic acid, panthenol and pantolactone). Within the context of this group panthenol and/or pantolactone is preferably used (see below). Derivatives of panthenol which can be used according to the invention are in particular the esters and ethers of panthenol as well as cationically derivatized panthenols. Individual representatives are for example panthenol triacetate, panthenol monoethyl ether and the monoacetate thereof as well as the cationic panthenol derivatives disclosed in WO 92/13829. The cited compounds of the vitamin B₅ type are preferably included in the agents according to the invention in amounts from 0.05 to 10 wt. %, relative to the total agent. Amounts from 0.1 to 5 wt. % are particularly preferred. Vitamin B6 (pyridoxine as well as pyridoxamine and pyridoxal).

Vitamin C (ascorbic acid). Vitamin C is used in the agents according to the invention preferably in amounts from 0.1 to 3 wt. %, relative to the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular esters such as acetate, nicotinate, phosphate and succinate, are preferably included in the agents according to the invention in amounts from 0.05 to 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S, 6aR)-2-oxohexahydrothienol[3,4-d]-imidazole-4-valeric acid, although this is now more widely known by the trivial name biotin. Biotin is preferably included in the agents according to the invention in amounts from 0.0001 to 1.0 wt. %, in particular in amounts from 0.001 to 0.01 wt. %.

In summary, hair treatment agents according to the invention are preferred which include—relative to their weight—0.1 to 5 wt. %, preferably 0.2 to 4 wt. %, particularly preferably 0.25 to 3.5 wt. %, more preferably 0.5 to 3 wt. % and in particular 0.5 to 2.5 wt. % of vitamins and/or provitamins and/or vitamin precursors, which are preferably assigned to groups A, B, C, E, F and H, wherein preferred agents include -2,4-dihydroxy-N-(3-hydroxypropyl)-3,3-dimethylbutyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂.

It has been found that certain quinones are particularly suitable as a care substance. As a further care substance the agents according to the invention can therefore include 0.0001 to 5 wt. % of at least one bioquinone of the formula (Ubi)

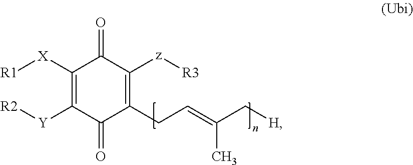

in which
X, Y, Z independently of one another denote —O— or —NH— or NR⁴— or a chemical bond
R¹, R², R³ independently of one another denote a hydrogen atom or an optionally substituted aryl group or an optionally substituted (C₁-C₆) alkyl group or a hydroxyalkyl group or a polyhydroxyalkyl group or an optionally substituted (C₁-C₆) alkylene group, or a (C₁-C₆) acyl residue, wherein preferred residues are selected independently of one another from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
R⁴ denotes —CH₃, —CH₂CH₃, —(CH₂)₂CH₂, CH(CH₃)₂, —(CH₂)₃CH₃, —CH(CH₃)CH₂CH₃, —CH₂CH(CH₃)₂, —C(CH₃)₃
n denotes values from 1 to 20, preferably 2 to 15, and in particular 5, 6, 7, 8, 9, 10.

Particularly preferred hair treatment agents according to the invention are characterized in that as a care substance they include—relative to their weight—0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein preferred agents include a ubiquinone of formula (Ubi)

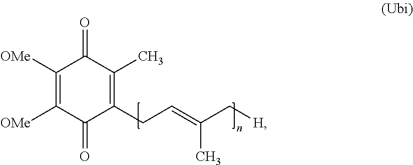

in which n denotes the values 6, 7, 8, 9 or 10, particularly preferably 10 (coenzyme Q10).

As an alternative to or in addition to the particularly preferred ubiquinones, the agents according to the invention can also include plastoquinones. Preferred agents according to the invention are characterized in that they include 0.0002 to 4 wt. %, preferably 0.0005 to 3 wt. %, particularly preferably 0.001 to 2 wt. %, more preferably 0.0015 to 1 and in particular 0.002 to 0.5 wt. % of at least one plastoquinone of the formula (Ubi-b)

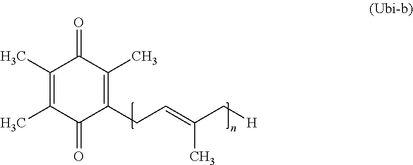

in which n denotes values from 1 to 20, preferably 2 to 15 and in particular 5, 6, 7, 8, 9, 10, wherein agents particularly preferably include plastoquinone PQ-9.

As a further care enhancer the agents according to the invention can include ectoine. Ectoine ((4S)-2-methyl-1,4,5,6-tetrahydropyrimidine-4-carboxylic acid) is a natural substance belonging to the group of compatible solutes. The highly water-binding, low-molecular-weight organic compound occurs in halophilic bacteria and allows these extremophile organisms to survive under stress conditions. Hair treatment agents that are preferred according to the invention are characterized in that they include—relative to their weight—0.001 to 10 wt. %, preferably 0.01 to 5 wt. %, particularly preferably 0.05 to 2.5 wt. % and in particular 0.1 to 1 wt. % of (S)-2-methyl-1,4,5,6-tetrahydro-4-pyrimidinecarboxylic acid (ectoine) and the physiologically acceptable salts of this compound and/or (S,S)-5-hydroxy-2-methyl-1,4,5,6-tetrahydro-4-pyrimidinecarboxylic acid (hydroxyectoine) and the physiologically acceptable salts of this compound.

To improve the elasticity and strength of the internal structure of hair treated with agents according to the invention, the agents according to the invention can include purine and/or purine derivatives as a care substance. In particular, the combination of purine and/or purine derivatives with ubiquinones and/or plastoquinones as a care substance means that hair treated with corresponding agents exhibits inter alia higher measured values in differential thermal analysis and improved wet and dry combability.

Purine (7H-imidazo[4,5-d]pyrimidine) does not occur freely in nature but forms the parent substance of purines. Purines for their part are a group of important compounds that occur widely in nature and are involved in human, animal, plant and microbial metabolic processes; they are derived from the parent substance by substitution with OH, NH₂, SH in the 2-, 6- and 8-position and/or with CH₃ in the 1-, 3-, 7-position. Purine can be produced for example from aminoacetonitrile and formamide. Purines and purine derivatives are often isolated from natural substances but are also available synthetically via many routes.

Preferred agents according to the invention include purine and/or purine derivatives in relatively narrow quantity ranges. Cosmetic agents that are preferred according to the invention are characterized in that they include—relative to their weight—0.001 to 2.5 wt. %, preferably 0.0025 to 1 wt. %, particularly preferably 0.005 to 0.5 wt. % and in particular 0.01 to 0.1 wt. % of purine(s) and/or purine derivative(s).

Some representatives of purine, purines and purine derivatives are particularly preferred according to the invention. Hair treatment agents that are preferred according to the invention are characterized in that as a care substance they include—relative to their weight—0.001 to 2.5 wt. %, preferably 0.0025 to 1 wt. %, particularly preferably 0.005 to 0.5 wt. % and in particular 0.01 to 0.1 wt. % of purine(s) and/or purine derivative(s), wherein preferred agents include purine and/or purine derivative(s) of the formula (Pur-I)

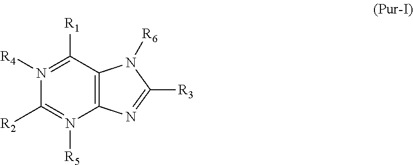

in which the residues R¹, R² and R³ are selected independently of one another from —H, —OH, NH₂, —SH and the residues R⁴, R⁵ and R⁶ are selected independently of one another from —H, —CH₃ and —CH₂—CH₃, the following compounds being preferred: purine (R¹═R²═R³═R⁴═R⁵═R⁶═H), adenine (R¹═NH₂, R²═R³═R⁴═R⁵═R⁶═H), guanine (R¹═OH, R²═NH₂, R³═R⁴═R⁵═R⁶═H), uric acid (R¹═R²═R³═OH, R⁴═R⁵═R⁶═H), hypoxanthine (R¹═OH, R²═R³═R⁴═R⁵═R⁶═H), 6-purinethiol (R¹═SH, R²═R³═R⁴═R⁵═R⁶═H), 6-thioguanine (R¹═SH, R²═NH₂, R³═R⁴═R⁵═R⁶═H), xanthine (R¹═R²═OH, R³═R⁴═R⁵═R⁶═H), caffeine (R¹═R²═OH, R³═H, R⁴═R⁵═R⁶═CH₃), theobromine (R¹═R²═OH, R³═R⁴═H, R⁵═R⁶═CH₃) theophylline (R¹═R²═OH, R³═H, R⁴═CH₃, R⁵═CH₃, R⁶═H).

It is furthermore advantageous to use purine or purine derivatives and bioquinones in a defined ratio to one another. Agents according to the invention are preferred here in which the weight ratio of purine (derivative(s)) and bioquinone(s) is 10:1 to 1:100, preferably 5:1 to 1:50, particularly preferably 2:1 to 1:20 and in particular 1:1 to 1:10.

As has already been mentioned, caffeine is a particularly preferred purine derivative, and coenzyme Q10 is a particularly preferred bioquinone. Particularly preferred agents according to the invention are therefore characterized in that they include—relative to their weight—0.001 to 2.5 wt. %, preferably 0.0025 to 1 wt. %, particularly preferably 0.005 to 0.5 wt.-% and in particular 0.01 to 0.1 wt. % of caffeine and 0.0002 to 4 wt. %, preferably 0.0005 to 3 wt. %, particularly preferably 0.001 to 2 wt. %, more preferably 0.0015 to 1 and in particular 0.002 to 0.5 wt. % of coenzyme Q10.

The agents according to the invention can also include flavonoids as a care substance. The flavonoids are a group of water-soluble plant dyes and they play an important role in the metabolism of many plants. Along with phenolic acids they belong to the polyphenols. There are well over 6500 different flavonoids known, and they can be divided into flavonols, flavones, flavanones, isoflavonoids and anthocyanins.

Flavonoids from all six groups can be used according to the invention, with certain representatives from the individual groups being preferred as a care substance because of their particularly intensive action. Preferred flavonols are quercetin, rutin, kaempferol, myricetin, isorhamnetin, preferred flavanols are catechin, gallocatechin, epicatechin, epigallocatechin gallate, theaflavin, thearubigin, preferred flavones are luteolin, apigenin, morin, preferred flavanones are hesperetin, naringenin, eriodictyol, preferred isoflavonoids are genistein, daidzein, and preferred anthocyanidins (anthocyanins) are cyanidin, delphinidin, malvidin, pelargonidin, peonidin, petunidin

Hair treatment agents that are particularly preferred according to the invention are characterized in that they include—relative to their weight—0.001 to 2.5 wt. %, preferably 0.0025 to 1 wt. %, particularly preferably 0.005 to 0.5 wt. % and in particular 0.01 to 0.1 wt. % of flavonoids, in particular flavonols, particularly preferably 3,3′,4′,5,7-pentahydroxyflavone (quercetin) and/or 3,3′,4′,5,7-pentahydroxyflavone-3-O-rutinoside (rutin).

Also preferred is the use of bisabolol and/or bisabolol oxides as a care substance in the agents according to the invention. Hair treatment agents according to the invention are preferred here which additionally include 0.001 to 5 wt. %, preferably 0.01 to 4 wt. %, particularly preferably 0.02 to 2.5 wt. % and in particular 0.1 to 1.5 wt. % of bisabolol and/or oxides of bisabolol, preferably (−)-alpha-bisabolol

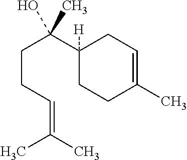 [SMILES: CC(C)=CCC[C@](C)(O)[C@H]1CC=C(C)CC1]

Creatine is also suitable according to the invention as a care substance. Creatine (3-methyl guanidinoacetic acid) is an organic acid which in vertebrates helps inter alia to supply energy to the muscles. Creatine is synthesized in the kidneys, the liver and the pancreas. It is formally derived from the amino acids glycine and arginine and 95% of it is present in the skeletal muscle. Particularly preferred hair treatment agents according to the invention include—relative to their weight—0.01 to 15 wt. %, preferably 0.025 to 12.5 wt. %, particularly preferably 0.05 to 10 wt. %, more preferably 0.1 to 7.5 wt. % and in particular 0.5 to 5 wt. % of N-methylguanidinoacetic acid (creatine).

The agents according to the invention can include in addition to the aforementioned ingredients and optional further ingredients further substances which prevent, alleviate or cure hair loss. A content of active ingredients which stabilize the hair root is advantageous in particular. These substances are described below:

Propecia (finasteride) is currently the only preparation that is approved worldwide and for which an effectiveness and tolerance has been proven in numerous studies. Propecia works by reducing the ability of DHT to form from testosterone.

Minoxidil with or without supplementary additives is probably the oldest demonstrably effective hair growth agent. For the treatment of hair loss it should be used for external application only. There are hair lotions including 2% to 5% minoxidil, also gels including up to 15% minoxidil. The effectiveness increases with the dose, but in hair lotions minoxidil is soluble only in a proportion of up to 5%. In many countries hair lotions including up to 2% minoxidil are available without a prescription.

Spironolactone in the form of a hair lotion and in combination with minoxidil can be used for external application to combat hormonal influences on the hair follicles. Spironolactone works as an androgen receptor blocker, in other words binding of DHT to the hair follicles is prevented.

In summary, hair treatment agents according to the invention are preferred which additionally include—relative to their weight—0.001 to 5 wt. % of hair root-stabilizing substances, in particular minoxidil and/or finasteride and/or ketoconazole.

In addition to the care substances the agents according to the invention can include further care substances. Their presence is not absolutely essential for achieving the effects according to the invention, but advanced effects, such as a pleasant feel or a pleasant application texture, can result from the use of these care substances.

As a further ingredient the agents according to the invention can include one or more amino acids to particular advantage. Amino acids that can particularly preferably be used according to the invention derive from the group comprising glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention include one or more amino acids in relatively narrow quantity ranges. Hair treatment agents that are preferred according to the invention are characterized in that as a care substance they include—relative to their weight—0.01 to 5 wt. %, preferably 0.02 to 2.5 wt. %, particularly preferably 0.05 to 1.5 wt. %, more preferably 0.075 to 1 wt. % and in particular 0.1 to 0.25 wt. % of amino acid(s), preferably from the group comprising glycine and/or alanine, and/or valine and/or lysine and/or leucine and/or threonine.

As a further constituent the agents according to the invention can include at least one carbohydrate from the group of monosaccharides, disaccharides and/or oligosaccharides. Hair treatment agents that are preferred according to the invention are characterized in that as a care substance they include—relative to their weight—0.01 to 5 wt. %, preferably 0.05 to 4.5 wt. %, particularly preferably 0.1 to 4 wt. %, more preferably 0.5 to 3.5 wt. % and in particular 0.75 to 2.5 wt. % of carbohydrate(s), selected from monosaccharides, disaccharides and/or oligosaccharides, wherein preferred carbohydrates are selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose
disaccharides, in particular sucrose and/or maltose and/or lactose and/or trehalose and/or cellobiose and/or gentiobiose and/or isomaltose.

As has already been mentioned, preferred agents according to the invention include (an) amino acid(s).

Amino acids that can particularly preferably be used according to the invention derive from the group comprising glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, B-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention include one or more amino acids in relatively narrow quantity ranges. Cosmetic agents that are preferred according to the invention are characterized in that they additionally include 0.05 to 5 wt. %, preferably 0.1 to 2.5 wt. %, particularly preferably 0.15 to 1 wt. % and in particular 0.2 to 0.5 wt. % of amino acid(s), preferably (an) amino acid(s) from the group comprising glycine and/or alanine, and/or valine and/or lysine and/or leucine and/or threonine.

Silicones are a particularly preferred group of ingredients.

Preferred agents according to the invention are characterized in that they include at least one silicone, preferably a silicone selected from:
(i) polyalkylsiloxanes, polyarylsiloxanes, polyalkylarylsiloxanes, which are volatile or non-volatile, straight-chain, branched or cyclic, crosslinked or uncrosslinked;
(ii) polysiloxanes which in their general structure include one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacyl amino groups;
   k) carboxyl groups
   l) sulfonic acid groups; and
   m) sulfate or thiosulfate groups;
(iii) linear polysiloxane(A)-polyoxyalkylene(B) block copolymers of the type (A-B)ₙ, where n>3;
(iv) grafted silicone polymers having a non-silicone-including, organic framework consisting of an organic main chain formed from organic monomers including no silicone, onto which at least one polysiloxane macromer has been grafted in the chain and optionally on at least one chain end;
(v) grafted silicone polymers having a polysiloxane framework, onto which non-silicone-including, organic monomers have been grafted, which have a polysiloxane main chain onto which at least one organic macromer including no silicone has been grafted in the chain and optionally on at least one of its ends;
   or mixtures thereof.

Agents that are particularly preferred according to the invention include the silicone(s) preferably in amounts from 0.1 to 10 wt. %, preferably from 0.25 to 7 wt. %, and in particular from 0.5 to 5 wt. %, relative in each case to the total agent.

Preferred silicones are described below.

Particularly preferred agents according to the invention are characterized in that they include at least one silicone of the formula Si-I

(CH₃)₃Si—[O—Si(CH₃₂]ₓ—O—Si(CH₃)₃ (Si-I),

in which x denotes a number from 0 to 100, preferably from 0 to 50, more preferably from 0 to 20 and in particular 0 to 10.

Under INCI nomenclature these silicones are known as DIMETHICONES. In the context of the present invention the compounds (CH₃)₃Si—O—Si(CH₃)₃, (CH₃)₃Si—O—(CH₃)₂Si—O—Si(CH₃)₃ and/or (CH₃)₃Si[O—(CH₃)₂Si]₂—O—Si(CH₃)₃ are particularly preferably used as the silicone of formula Si-I.

Mixtures of the aforementioned silicones can of course also be included in the agents according to the invention.

Preferred silicones for use according to the invention have viscosities at 20° C. of 0.2 to 2 mm²s⁻¹, silicones having viscosities of 0.5 to 1 mm²s⁻¹ being particularly preferred.

Most particularly preferred hair treatment agents according to the invention are characterized in that they include—relative to their weight—0.1 to 10 wt. %, preferably 0.15 to 7.5 wt. %, more preferably 0.2 to 5 wt. %, still more preferably 0.25 to 2.5 wt. % and in particular 0.4 to 1.5 wt. % of polydimethylsiloxane (INCI name: dimethicone).

Hair treatment agents according to the invention that are preferred in particular are characterized in that they include, relative to their weight,
a) 0.5 to 2.5 wt. % of Behenoyl PG Trimonium Chloride,
b) 0.5 to 2.5 wt. % of Behentrimonium Chloride,
c) 0.5 to 2.5 wt. % of Quaternium-87,
d) 0.5 to 2.5 wt. % of Dimethylacrylamide/Ethyltrimonium Chloride Methacrylate Copolymer,
e) 0.5 to 2.5 wt. % of isopropyl myristate,
f) 0.4 to 1.5 wt. % of dimethicone.

Particularly preferred agents according to the invention include one or more amino-functional silicones. Such silicones can be described for example by the formula

M(RₐQ_{b}SiO_{(4-a-b)/2)x}(R_{c}SiO_{(4-c)/2)y}M

wherein in the above formula R is a hydrocarbon or a hydrocarbon residue having 1 to approximately 6 carbon atoms, Q is a polar residue of the general formula —R¹HZ, in which R¹ is a divalent linking group which is bound to hydrogen and the residue Z, composed of carbon and hydrogen atoms, carbon, hydrogen and oxygen atoms or carbon, hydrogen and nitrogen atoms, and Z is an organic amino-functional residue including at least one amino-functional group; “a” assumes values in the range from approximately 0 to approximately 2, “b” assumes values in the range from approximately 1 to approximately 3, “a” +“b” is less than or equal to 3, and “c” is a number in the range from approximately 1 to approximately 3, and x is a number in the range from 1 to approximately 2000, preferably from approximately 3 to approximately 50 and most preferably from approximately 3 to approximately 25, and y is a number in the range from approximately 20 to approximately 10,000, preferably from approximately 125 to approximately 10,000 and most preferably from approximately 150 to approximately 1000, and M is a suitable silicone end group, as is known in the prior art, preferably trimethylsiloxy. Non-restricting examples of the residues represented by R include alkyl residues such as methyl, ethyl, propyl, isopropyl, isopropyl, butyl, isobutyl, amyl, isoamyl, hexyl, isohexyl and similar; alkenyl residues, such as vinyl, halovinyl, alkyl vinyl, allyl, haloallyl, alkyl allyl; cycloalkyl residues, such as cyclobutyl, cyclopentyl, cyclohexyl and similar; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and similar, and sulfur-including residues, such as mercaptoethyl, mercaptopropyl, mercaptohexyl, mercaptophenyl and similar; R is preferably an alkyl residue including 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—.
Z is an organic, amino-functional residue including at least one functional amino group. A possible formula for Z is NH(CH₂)_{z}NH₂, in which z is 1 or more. Another possible formula for Z is —NH(CH₂)_{z}(CH ₂)_{zz}NH, in which both z and zz are independently 1 or more, this structure comprising diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.
Q is most preferably a polar, amine-functional residue of the formula —CH₂CH₂CH₂NHCH₂CH₂NH₂. In the formulae “a” assumes values in the range from approximately 0 to approximately 2, “b” assumes values in the range from approximately 2 to approximately 3, “a” +“b” is less than or equal to 3, and “c” is a number in the range from approximately 1 to approximately 3. The molar ratio of the RₐQ_{b}SiO_{(4-a-b)/2} units to the R_{c}SiO_{(4-c)/2} units is in the range from approximately 1:2 to 1:65, preferably from approximately 1:5 to approximately 1:65 and most preferably from approximately 1:15 to approximately 1:20. If one or more silicones of the above formula is used, then the different variable substituents in the above formula can differ in the different silicone components that are present in the silicone mixture.

Particularly preferred agents according to the invention are characterized in that they include at least one amino-functional silicone of the formula (Si-IIa)

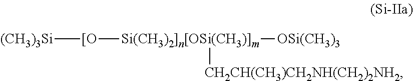

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10.

Under the INCI declaration these silicones are known as trimethylsilylamodimethicones. Agents according to the invention are also particularly preferred which include an amino-functional silicone of the formula (Si-IIb)

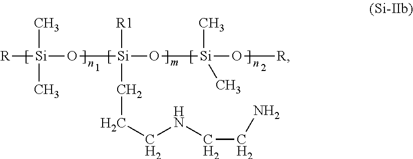

in which R denotes —OH, —O—CH₃ or a —CH₃ group and m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicones.

Irrespective of which amino-functional silicones are used, agents according to the invention are preferred which include an amino-functional silicone whose amine value is above 0.25 meq/g, preferably above 0.3 meq/g and in particular above 0.4 meq/g. The amine value denotes the milli-equivalents of amine per gram of the amino-functional silicone. It can be determined by titration and also specified in the unit mg KOH/g.

Agents that are preferred according to the invention are characterized in that they include, relative to their weight, 0.01 to 10 wt. %, preferably 0.1 to 8 wt. %, particularly preferably 0.25 to 7.5 wt. % and in particular 0.5 to 5 wt. % of amino-functional silicone(s).

The cyclic dimethicones referred to under INCI as CYCLOMETHICONES can also be used to advantage according to the invention. Agents according to the invention are preferred here which include at least one silicone of the formula Si-III

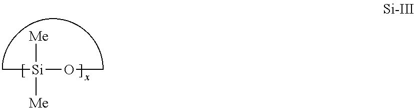

in which x denotes a number from 3 to 200, preferably from 3 to 10, more preferably from 30 to 7 and in particular 3, 4, 5 or 6.

The silicones described above have a backbone which is made up of —Si—O—Si units. These Si—O—Si units can of course also be interrupted by carbon chains. Corresponding molecules can be obtained by chain extension reactions and are preferably used in the form of silicone-in-water emulsions.

Agents that are likewise preferred according to the invention are characterized in that they include at least one silicone of the formula Si-IV

R₃Si—[O—SiR₂]ₓ—(CH₂)ₙ—[O—SiR₂]_{y}—O—SiR₃ (Si-IV),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

The silicones are by preference water-soluble. Agents that are preferred according to the invention are characterized in that they include at least one water-soluble silicone.

For aesthetic reasons “clear” products are often preferred by consumers. Hair treatment agents that are preferred according to the invention are therefore characterized in that they are transparent or translucent.

Transparent or translucent within the context of the present invention is understood to be a composition having an NTU value of below 100. The NTU unit (nephelometric turbidity unit) is a unit for turbidity measurements in liquids that is used in water treatment. It is the unit for a turbidity of a liquid measured with a calibrated nephelometer.

In a preferred embodiment of the invention an agent according to the invention can also include UV filters (I). There are no general restrictions on the UV filters to be used according to the invention in terms of their structure and their physical properties. In fact all UV filters that can be used in the cosmetics sector whose absorption maximum is in the UVA (315-400 nm), UVB (280-315 nm) or UVC (<280 nm) range are suitable. UV filters having an absorption maximum in the UVB range, in particular in the range from approximately 280 to approximately 300 nm, are particularly preferred.

The UV filters used according to the invention can be selected for example from substituted benzophenones, p-aminobenzoic acid esters, diphenyl acrylic acid esters, cinnamic acid esters, salicylic acid esters, benzimidazoles and o-aminobenzoic acid esters.

Examples of UV filters that can be used according to the invention are 4-aminobenzoic acid, N,N,N-trimethyl-4-(2-oxoborn-3-ylidene methyl)aniline methyl sulfate, 3,3,5-trimethyl cyclohexyl salicylate (Homosalate), 2-hydroxy-4-methoxybenzophenone (Benzophenone-3; Uvinul®M 40, Uvasorb®MET, Neo Heliopan BB, Eusolex 4360), 2-phenylbenzimidazole-5-sulfonic acid and potassium, sodium and triethanolamine salts thereof (Phenylbenzimidazole sulfonic acid; Parsol®HS; Neo Heliopan®Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (Butyl methoxydibenzoylmethane; Parsol®1789, Eusolex®9020), α-(2-oxobom-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (Octyl Dimethyl PABA; Uvasorb®DMO, Escalol®507, Eusolex®6007), salicylic acid-2-ethylhexyl ester (Octyl Salicylate; Escalol®587, Neo Heliopan®OS, Uvinul®018), 4-methoxycinnamic acid isopentyl ester (Isoamyl p-Methoxycinnamate; Neo Heliopan®E 1000), 4-methoxycinnamic acid-2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene camphor; Parsol®5000, Eusolex®6300), 3-benzylidene camphor (3-Benzylidene camphor), 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide, 2,4-dihydroxybenzophenone (Benzophenone-1; Uvasorb®20 H, Uvinul®400), 1,1′-diphenylacrylonitrilic acid-2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid-2′-ethylhexyl ester.

4-Aminobenzoic acid, N,N,N-trimethyl-4-(2-oxoborn-3-ylidene methyl)aniline methyl sulfate, 3,3,5-trimethyl cyclohexyl salicylate, 2-hydroxy-4-methoxybenzophenone, 2-phenylbenzimidazole-5-sulfonic acid and potassium, sodium and triethanolamine salts thereof, 3,3 ‘-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxobom-3-ylidene)toluene-4-sulfonic acid and salts thereof, ethoxylated 4-aminobenzoic acid ethyl ester, 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof, 3-(4’-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)[2-oxobom-3-ylidene methyl]benzyl}acrylamide are preferred. Most particularly preferred according to the invention are 2-hydroxy-4-methoxybenzophenone, 2-phenylbenzimidazole-5-sulfonic acid and potassium, sodium and triethanolamine salts thereof, 1-(4-tert-butylphenyl)-3-(4-methoxyphenyl)propane-1,3-dione, 4-methoxycinnamic acid-2-ethylhexyl ester and 3-(4′-methylbenzylidene)-D,L-camphor.

UV filters whose molar extinction coefficient at the absorption maximum is above 15,000, in particular above 20,000, are preferred.

It has moreover been found that with structurally similar UV filters, the water-insoluble compound has in many cases the greater effect in the context of the teaching according to the invention as compared with water-soluble compounds that differ therefrom by one or more additional ionic groups. Within the context of the invention water-insoluble is understood to mean UV filters that dissolve in water at 20° C. by no more than 1 wt. %, in particular no more than 0.1 wt. %. These compounds should furthermore be soluble in conventional cosmetic oil components at room temperature by at least 0.1, in particular at least 1 wt. %. The use of water-insoluble UV filters can therefore be preferred according to the invention.

According to a further embodiment of the invention UV filters having a cationic group, in particular a quaternary ammonium group, are preferred.

These UV filters have the general structure U-Q.

The structural part U denotes a group that absorbs UV radiation. This group can in principle be derived from the aforementioned known UV filters that are suitable for use in the cosmetic sector by substituting a group, generally a hydrogen atom, of the UV filter with a cationic group Q, in particular having a quaternary amino function.

Compounds from which the structural part U can be derived are for example substituted benzophenones, p-aminobenzoic acid esters, diphenyl acrylic acid esters, cinnamic acid esters, salicylic acid esters, benzimidazoles and o-aminobenzoic acid esters.

Structural parts U that derive from cinnamic acid amide or from N,N-dimethylaminobenzoic acid amide are preferred according to the invention.

The structural parts U can in principle be chosen such that the absorption maximum of the UV filters can lie in both the UVA range (315-400 nm) and in the UVB range (280-315 nm) or the UVC range (<280 nm). UV filters having an absorption maximum in the UVB range, in particular in the range from approximately 280 to approximately 300 nm, are particularly preferred.

Depending also on the structural part Q, the structural part U is furthermore preferably chosen such that the molar extinction coefficient of the UV filter at the absorption maximum is above 15,000, in particular above 20,000.

The structural part Q preferably includes a quaternary ammonium group as the cationic group. This quaternary ammonium group can in principle be linked directly to the structural part U, such that the structural part U is one of the four substituents of the positively charged nitrogen atom. However, one of the four substituents at the positively charged nitrogen atom is preferably a group, in particular an alkylene group having 2 to 6 carbon atoms, that functions as a link between the structural part U and the positively charged nitrogen atom.

The group Q advantageously has the general structure —(CH₂)ₓ—N⁺R¹R²R³ x⁻, in which x denotes an integer from 1 to 4, R¹ and R² independently of one another denote C₁₋₄ alkyl groups, R³ denotes a C₁₋₂₂ alkyl group or a benzyl group and X⁻ denotes a physiologically acceptable anion. In the context of this general structure x preferably denotes the number 3, R¹ and R² each denote a methyl group and R³ denotes either a methyl group or a saturated or unsaturated, linear or branched hydrocarbon chain having 8 to 22, in particular 10 to 18, carbon atoms.

Physiologically acceptable anions are for example inorganic anions such as halides, in particular chloride, bromide and fluoride, sulfate ions and phosphate ions as well as organic anions such as lactate, citrate, acetate, tartrate, methosulfate and tosylate.

Two preferred UV filters having cationic groups are the compounds cinnamic acid amidopropyl trimethylammonium chloride (lncroquat®UV-283) and dodecyl dimethylaminobenzamidopropyl dimethylammonium tosylate (Escalol® HP 610), which are available as commercial products.

The teaching according to the invention naturally also comprises the use of a combination of a plurality of UV filters. In the context of this embodiment the combination of at least one water-insoluble UV filter with at least one UV filter having a cationic group is preferred.

The UV filters (I) are conventionally included in the agents according to the invention in amounts from 0.1 to 5 wt. %, relative to the total agent. Amounts from 0.4 to 2.5 wt. % are preferred.

The agents according to the invention can moreover include a 2-pyrrolidinone-5-carboxylic acid and derivatives thereof (J). The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents according to the invention are preferably 0.05 to 10 wt. %, relative to the total agent, particularly preferably 0.1 to 5, and in particular 0.1 to 3 wt. %.

It can additionally prove advantageous if penetration auxiliaries and/or swelling agents (M) are included in the agents according to the invention. They include for example urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

Within the meaning of the invention short-chain carboxylic acids (N) can advantageously additionally support the active ingredient complex (A). Short-chain carboxylic acids and derivatives thereof are understood within the meaning of the invention to be carboxylic acids which can be saturated or unsaturated and/or straight-chain or branched or cyclic and/or aromatic and/or heterocyclic and which have a molecular weight of less than 750. Saturated or unsaturated straight-chain or branched carboxylic acids having a chain length of 1 to 16 C atoms in the chain can be preferred within the meaning of the invention, with those having a chain length of 1 to 12 C atoms in the chain being most particularly preferred.

Within the meaning of the invention the short-chain carboxylic acids can have one, two, three or more carboxyl groups. Carboxylic acids having more than one carboxyl group, in particular di- and tricarboxylic acids, are preferred within the meaning of the invention. The carboxyl groups can be present wholly or partially as the ester, acid anhydride, lactone, amide, imidic acid, lactam, lactim, dicarboximide, carbohydrazide, hydrazone, hydroxam, hydroxime, amidine, amidoxime, nitrile, phosphonic ester or phosphate ester. The carboxylic acids used according to the invention can of course be substituted along the carbon chain or the ring skeleton. The substituents of the carboxylic acids used according to the invention include for example C₁-C₈ alkyl, C₂-C₈ alkenyl, aryl, aralkyl and aralkenyl, hydroxymethyl, C2-C₈ hydroxyalkyl, C2-C₈ hydroxyalkenyl, aminomethyl, C₂-C₈ aminoalkyl, cyano, formyl, oxo, thioxo, hydroxyl, mercapto, amino, carboxyl or imino groups. Preferred substituents are C₁-C₈ alkyl, hydroxymethyl, hydroxyl, amino and carboxyl groups. Substituents in the α-position are particularly preferred. Most particularly preferred substituents are hydroxyl, alkoxy and amino groups, wherein the amino function can optionally be further substituted by alkyl, aryl, aralkyl and/or alkenyl residues. Preferred carboxylic acid derivatives are moreover likewise phosphonic and phosphate esters.

In a further preferred embodiment the agents according to the invention can include emulsifiers (F). Emulsifiers cause water-stable or oil-stable adsorption layers, which protect the dispersed droplets against coalescence and thus stabilize the emulsion, to form at the phase interface. Like surfactants, emulsifiers are therefore made up of a hydrophobic and a hydrophilic molecule part. Hydrophilic emulsifiers preferably form O/W emulsions and hydrophobic emulsifiers preferably form W/O emulsions. An emulsion is understood to be a distribution in droplet form (dispersion) of one liquid in another liquid, expending energy to create stabilizing phase interfaces by means of surfactants. The choice of these emulsifying surfactants or emulsifiers is governed by the substances to be dispersed and the external phase in each case as well as by the fine-particle character of the emulsion. Emulsifiers that can be used according to the invention are for example
addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group,
C₁₂-C₂₂ fatty acid monoesters and diesters of addition products of 1 to 30 mol of ethylene oxide with polyols having 3 to 6 carbon atoms, in particular with glycerol,
ethylene oxide and polyglycerol addition products with methyl glucoside fatty acid esters, fatty acid alkanol amides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as the sugar component are preferred,
mixtures of alkyl (oligo)glucosides and fatty alcohols, for example the commercially available product Montanov®68,
addition products of 5 to 60 mol of ethylene oxide with castor oil and hydrogenated castor oil,
partial esters of polyols having 3 to 6 carbon atoms with saturated fatty acids having 8 to 22 C atoms,
sterols. Sterols are understood to be a group of steroids which bear a hydroxyl group on C atom 3 of the steroid skeleton and are isolated from both animal tissue (zoosterols) and vegetable fats (phytosterols). Examples of zoosterols are cholesterol and lanosterol. Examples of suitable phytosterols are ergosterol, stigmasterol and sitosterol. Sterols known as mycosterols are also isolated from fungi and yeasts,
phospholipids. These are understood above all to be the glucose phospholipids which are obtained for example as lecithins or phosphatidyl cholines from for example egg yolk or plant seeds (e.g. soybeans),
fatty acid esters of sugars and sugar alcohols, such as sorbitol,
polyglycerols and polyglycerol derivatives such as for example polyglycerol poly-12-hydroxystearate (commercial product Dehymuls® PGPH),
linear and branched fatty acids having 8 to 30 C atoms and Na, K, ammonium, Ca, Mg and Zn salts thereof.

The agents according to the invention include emulsifiers preferably in amounts from 0.1 to 25 wt. %, in particular 0.5 to 15 wt. %, relative to the total agent.

The compositions according to the invention can preferably include at least one non-ionogenic emulsifier having an HLB value of 8 to 18. Non-ionogenic emulsifiers having an HLB value of 10 to 15 can be particularly preferred according to the invention.

It has moreover proved advantageous if the agents according to the invention include further polymers, preferably anionic and/or non-ionic polymers.

The anionic polymers (G2) are anionic polymers having carboxylate and/or sulfonate groups. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid.

Anionic polymers including as the sole monomer or as a co-monomer 2-acrylamido-2-methylpropane sulfonic acid, in which some or all of the sulfonic acid group can be present as the sodium, potassium, ammonium, mono- or triethanolammonium salt, have proved to be most particularly effective.

The homopolymer of 2-acrylamido-2-methylpropane sulfonic acid, which is available commercially for example under the name Rheothik®11-80, is particularly preferred.

Within this embodiment it can be preferable to use copolymers consisting of at least one anionic monomer and at least one non-ionogenic monomer. With regard to the anionic monomers reference is made to the aforementioned substances. Preferred non-ionogenic monomers are acrylamide, methacrylamide, acrylic acid ester, methacrylic acid ester, vinyl pyrrolidone, vinyl ether and vinyl ester.

Preferred anionic copolymers are acrylic acid-acrylamide copolymers as well as in particular polyacrylamide copolymers with monomers including sulfonic acid groups. A particularly preferred anionic copolymer consists of 70 to 55 mol % of acrylamide and 30 to 45 mol % of 2-acrylamido-2-methylpropane sulfonic acid, wherein some or all of the sulfonic acid group is present as the sodium, potassium, ammonium, mono- or triethanolammonium salt. This copolymer can also be crosslinked, wherein polyolefinically unsaturated compounds such as tetraallyl oxyethane, allyl sucrose, allyl pentaerythritol and methylene bisacrylamide are preferably used as crosslinking agents. Such a polymer is included in the commercial product Sepigel® 305 from SEPPIC. The use of this compound, which in addition to the polymer component includes a mixture of hydrocarbons (C₁₃-C₁₄ isoparaffin) and a non-ionogenic emulsifier (Laureth-7), has proved to be particularly advantageous within the context of the teaching according to the invention.

The sodium acryloyldimethyltaurate copolymers sold under the name Simulgel® 600 as a compound with isohexadecane and Polysorbate-80 have also proved to be particularly effective according to the invention.

Likewise preferred anionic homopolymers are uncrosslinked and crosslinked polyacrylic acids. Allyl ethers of pentaerythritol, of sucrose and of propylene can be preferred crosslinking agents here. Such compounds are available commercially for example under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

In a further embodiment the agents according to the invention can include non-ionogenic polymers (G4).

Suitable non-ionogenic polymers are for example:
vinyl pyrrolidone/vinyl ester copolymers, such as are sold for example under the trademark Luviskol® (BASF). Luviskol® VA 64 and Luviskol® VA 73, both of which are vinyl pyrrolidone/vinyl acetate copolymers, are likewise preferred non-ionic polymers;
cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules);
starch and derivatives thereof, in particular starch ethers, for example Structure® XL (National Starch), a multifunctional, salt-tolerant starch;
shellac;
polyvinyl pyrrolidones, such as are sold for example under the name Luviskol® (BASF);
siloxanes. These siloxanes can be both water-soluble and water-insoluble. Both volatile and non-volatile siloxanes are suitable, wherein compounds whose boiling point under normal pressure is above 200° C. are understood to be non-volatile siloxanes. Preferred siloxanes are polydialkyl siloxanes, such as for example polydimethyl siloxane, polyalkylaryl siloxanes, such as for example polyphenylmethyl siloxane, ethoxylated polydialkyl siloxanes as well as polydialkyl siloxanes including amine and/or hydroxyl groups;
glycoside-substituted silicones.

It is also possible according to the invention for the preparations to include a plurality of, in particular two, different polymers of the same charge and/or an ionic and an amphoteric and/or non-ionic polymer.

The further polymers (G) are preferably included in the agents according to the invention in amounts from 0.05 to 10 wt. %, relative to the total agent. Amounts from 0.1 to 5, in particular from 0.1 to 3 wt. %, are particularly preferred.

With regard to further optional components and to the amounts of these components used, reference is expressly made to the relevant manuals known to the person skilled in the art.

As already mentioned, importance is attached to the good care effect of the agents according to the invention in particular because they deliver outstanding results even in the presence of oxidizing agents—for example in the context of oxidative hair coloring.

The present invention also provides a method for treating keratinic fibers, in particular human hair, wherein a hair treatment agent according to the invention is applied to keratinic fibers and either left there until the next hair wash (“leave-on” product) or rinsed off after a contact time from 30 to 300 seconds (“rinse-off” product).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent, comprising:
a) at least one quaternary ammonium compound according to formula I 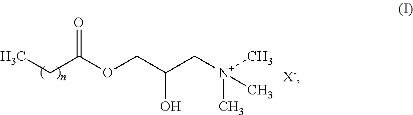
in which n denotes an integer from 10 to 24 and X⁻ denotes an anion,
b) at least one quaternary ammonium compound according to formula II 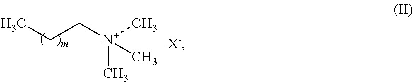
in which m denotes an integer from 10 to 24 and X⁻ denotes an anion,
c) at least one quaternary ammonium compound of formula III 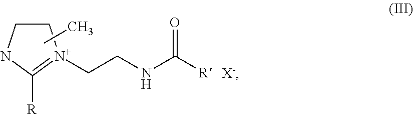
in which R and R′ independently of one another each denote a saturated or unsaturated, linear or branched hydrocarbon residue having a chain length of 16 to 30 carbon atoms and X⁻ denotes an anion, and
d) at least one cationic copolymer, including monomers of formula (IV) and monomers of formula (V) 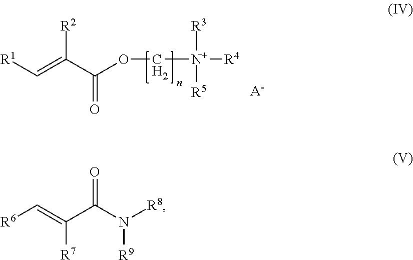
in which R¹ to R⁹ independently of one another denote hydrogen, C₁₋₄ alkyl such as methyl, ethyl, propyl, isopropyl, butyl, isobutyl or tert-butyl, with the proviso that at least one of the residues R⁶, R⁷, R⁸ or R⁹ denotes C₁₋₄ alkyl, n denotes integers from 1 to 8 and A⁻ denotes a physiologically acceptable anion.

2. The hair treatment agent according to claim 1, characterized in that it additionally includes—relative to its weight—0.1 to 20 wt. % of isopropyl myristate.

3. The hair treatment agent according to claim 1, characterized in that it includes—relative to its weight—0.1 to 20 wt. % of at least one quaternary ammonium compound of formula I in which n denotes the number 20 and A⁻ denotes chloride (INCI name: Behenoyl PG Trimonium Chloride).

4. The hair treatment agent according to claim 1, characterized in that it includes—relative to its weight—0.1 to 15 wt. % of at least one quaternary ammonium compound of formula II in which n denotes the number 20 and A⁻ denotes chloride (INCI name: Behentrimonium Chloride).

5. The hair treatment agent according to claims 1, characterized in that it includes—relative to its weight—0.1 to 15 wt. % of at least one quaternary ammonium compound of formula III in which R and R′ independently of one another are selected from stearyl and oleoyl residues and A″ denotes methosulfate (INCI name: Quaternium-87).

6. The hair treatment agent according to claim 1, characterized in that it includes—relative to its weight—0.1 to 15 wt. % of at least one cationic copolymer including monomers of formula (IV) and monomers of formula (V), in which R¹═R⁶═R⁷═—H and R²═R³═R⁴═R⁵═R⁸═R⁹═—CH₃, n denotes the number 2 and A⁻ denotes chloride (INCI name: Dimethylacrylamide/Ethyltrimonium Chloride Methacrylate Copolymer).

7. The hair treatment agent according to claim 1, characterized in that it additionally includes—relative to its weight—0.1 to 10 wt. % of polydimethylsiloxane (INCI name: dimethicone).

8. The hair treatment agent according to claim 1, including, relative to its weight,
a) 0.5 to 2.5 wt. % of Behenoyl PG Trimonium Chloride,
b) 0.5 to 2.5 wt. % of Behentrimonium Chloride,
c) 0.5 to 2.5 wt. % of Quaternium-87, and
d) 0.5 to 2.5 wt. % of Dimethylacrylamide/Ethyltrimonium Chloride Methacrylate Copolymer.

9. The hair treatment agent according to claim 1, including, relative to its weight,
a) 0.5 to 2.5 wt. % of Behenoyl PG Trimonium Chloride,
b) 0.5 to 2.5 wt. % of Behentrimonium Chloride,
c) 0.5 to 2.5 wt. % of Quaternium-87,
d) 0.5 to 2.5 wt. % of Dimethylacrylamide/Ethyltrimonium Chloride Methacrylate Copolymer, and
e) 0.5 to 2.5 wt. % of isopropyl myristate.

10. The hair treatment agent according to claim 1, including, relative to its weight,
a) 0.5 to 2.5 wt. % of Behenoyl PG Trimonium Chloride,
b) 0.5 to 2.5 wt. % of Behentrimonium Chloride,
c) 0.5 to 2.5 wt. % of Quaternium-87,
d) 0.5 to 2.5 wt. % of Dimethylacrylamide/Ethyltrimonium Chloride Methacrylate Copolymer,
e) 0.5 to 2.5 wt. % of isopropyl myristate, and
f) 0.4 to 1.5 wt. % of dimethicone.
